SEQUENCE LISTING (1 sequence)

SEQ ID NO: 1 - protein, 604 aa, Homo sapiens
  ref <308>: DATABASE ACCESSION NUMBER: NP_004813
  ref <309>: DATABASE ENTRY DATE: 2006-11-17
  ref <313>: RELEVANT RESIDUES IN SEQ ID NO: (1)..(604)
MMRAVWEALAALAAVACLVGAVRGGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGYYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKINMKKYCKKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

(19) United States
(12) Patent Application Publication
(10) Pub. No.: US 2016/0369242 A1
(43) Pub. Date: Dec. 22, 2016

(54) METHOD FOR PRODUCING INDUCED PLURIPOTENT CELLS

(71) Applicants: CENTRE LEON BERARD, LYON (FR); UNIVERSITE CLAUDE BERNARD LYON 1, Villeurbanne (FR)

(72) Inventors: Fabrice LAVIAL, Thurins (FR); Patrick MEHLEN, Serezin de la Tour (FR); Agnès BERNET, Genas (FR)

(73) Assignees: CENTRE LEON BERARD, Lyon (FR); UNIVERSITE CLAUDE BERNARD LYON 1, VILLEURBANNE (FR); INSTITUT NATIONAL DE LA SANTE ET DE LA RECHERCHE M EDICALE (INSERM), PARIS (FR)

(21) Appl. No.: 15/110,979

(22) PCT Filed: Jan. 13, 2015
(86) PCT No.: PCT/EP2015/050478

§ 371 Date: Jul. 12, 2016

(30) Foreign Application Priority Data
Jan. 13, 2014 (FR) 1450252

(51) Int. Cl. C12N 5/074

(52) U.S. Cl. CPC C12N 5/0696; C12N 2501/602; C12N 2501/604; C12N 2501/606; C12N 2501/603; C12N 2506/1307; C12N 2501/998

(57) ABSTRACT

The invention relates to a method for producing induced pluripotent stem cells (iPS) by culturing somatic cells subjected to a cellular reprogramming method, characterised in that the somatic cells are cultured in the presence of netrin-1 or an analogue of netrin-1 at least at the beginning of the cellular reprogramming method.

### FIELD OF THE INVENTION

The present invention concerns a method for producing induced pluripotent cells (iPSCs) that has improved efficiency and gives improved homogeneity of the iPS cells obtained. This improvement is obtained by using Netrin-1 or an analogue of Netrin-1 to prevent or block cell death mediated by DCC (Deleted in Colorectal Carcinoma) or UNC5s (UNC5A, UNC5B, UNC5C and UNC5D) receptors of Netrin-1 in the initial phases of the cell reprogramming process.

### STATE OF THE ART

At the present time much research has been reported showing that somatic cells can be de-differentiated into cells called induced pluripotent stem cells or iPS cells which exhibit similar characteristics to embryonic stem cells.

The generating of these induced pluripotent stem cells (iPS cells or iPSCs) is highly promising for regenerative medicine.

Cell de-differentiation or reprogramming methods generally entail the expressing of one or more key exogenous factors allowing reprogramming of somatic cells to maintain stem cell pluripotency. Cell reprogramming methods, whereby somatic cells are cultured on a suitable culture medium so that they become and remain pluripotent through the expression of different transcription factors, have been largely described in the literature, in particular through the expression of factors in the Oct, Sox, Klf and Myc family, and more particularly the use of a Oct4, Sox2, Klf4 and c-Myc mixture or OSKM cocktail.

These cell reprogramming methods are well known to the person skilled in the art. For a review, particular mention is made of the following reference: Gonzalez et al., Nat Rev Genet 2011, 12:231-242.

However, these methods prove to be stochastic and scarcely efficient and the development of therapeutic applications of iPSCs is limited by the low efficacy of reprogramming methods and the heterogeneity of results.

The inventors, by studying induced cell death mechanisms throughout this cell reprogramming process, have been able to evidence the importance of the role played by Netrin-1 and the DCC and UNC5s receptors thereof, DCC in particular, in somatic cells subjected to this reprogramming to iPS cells.

Netrin-1 or Ntn1 is a diffusible secreted protein related to laminin. Netrin-1 is the ligand of DCC (Deleted in Colorectal Cancer) and UNC5A-D (UNCoordinated 5 A, B, C and D) receptors. Historically, Netrin-1 (from Sanskrit: one who guides”) was the first axonal guiding factor characterized for its role in targeting the axons of commissural neurons of the spinal cord (Serafini et al., 1994; Kennedy et al., 2004; Serafini et al., 1996). Its key role in controlling axon guidance and neuron migration was later confirmed in other territories of the nerve system (Moore et al., 2007). More recently, different articles have established the role of Netrin-1 and its receptors in the control of angiogenesis (Lu et al., 2004; Park et al., 2004; Wilson et al., 2006; Larrivee et al. 2007; Navankasatussas et al., 2008; Ahmed et al., 2010; Epting et al., 2010) or tumorigenesis (Mazelin et al., 2004; Fitamant et al., 2008). Its involvement in the control of tumorigenesis was evidenced by the fact that Netrin-1, by binding to its DCC or UNC5s receptors, blocks the pro-apoptotic action of these non-bonded receptors (Mehlen and Goldschneider, Oncogene, 2010; Mehlen et al., Nature Review Cancer, 2011).

### DISCLOSURE OF THE INVENTION

The subject of the present invention is a method for producing induce pluripotent stem cells (iPSCs) via culture of somatic cells subjected to a cell reprogramming process, characterized in that the somatic cells are cultured in the presence of Netrin-1 or an analogue of Netrin-1 at least at the start of the cell reprogramming process.

A further subject of the present invention is the use of Netrin-1 or analogue of Netrin-1 to produce induced pluripotent stem cells (iPSCs) via culture of somatic cells subjected to a cell reprogramming process, in particular to improve the efficacy of cell reprogramming of somatic cells to iPS cells.

### DETAILED DESCRIPTION OF THE INVENTION

The invention does not concern the identification of novel systems for the reprogramming of somatic cells to iPSCs. The invention concerns the inhibition of cell death induced by the reprogramming process by acting on the interaction of the DCC or UNC5s receptors of somatic cells with Netrin-1 or an analogue of Netrin-1.

The inventors have been able to evidence that during the cell reprogramming process the expression of Netrin-1 is rapidly reduced whereas the expression of its DCC receptor is maintained, a situation leading to DCC-mediated cell death.

The object of the invention is therefore to compensate for this reduction in Netrin-1 in the culture medium at the start of the reprogramming process, at the time when its expression decreases.

The invention therefore concerns the culturing somatic cells in the progress of cell reprogramming in the presence of Netrin-1 in the culture medium, or an analogue of Netrin-1, to prevent or block cell death mediated by the DCC or UNC5s receptors in the somatic cells when they are being reprogrammed.

It has the advantage of being simple to implement since it uses a soluble molecule, Netrin-1 or an analogue of Netrin-1, without requiring addition genomic integration, to prevent of block cell death induced by the reprogramming process of somatic cells to iPS cells.

According to a first aspect, the present invention therefore concerns an in vitro method for producing induced pluripotent stem cells (iPSCs) via culture of somatic cells subjected to a cell reprogramming process, characterized in that the somatic cells are cultured in the presence of Netrin-1 or an analogue of Netrin-1 at least at the start of the cell reprogramming process.

The present invention therefore covers an in vitro method to prepare induced pluripotent stem cells (iPSCs) which comprises the following steps:
culturing somatic cells;
subjecting these cells to a cell reprogramming process in a culture medium comprising Netrin-1 or an analogue of Netrin-1 at least at the start of the reprogramming process.

In the invention by “induced pluripotent stem cells” or “iPSCs” is meant cells obtained by in vitro cell reprogramming of somatic cells to which stem cell pluripotency factors have been added.

Cell reprogramming refers to a process of de-differentiating somatic cells to pluripotent stem cells.

The inventors have been able to show first that the presence of Netrin-1 increases the efficacy of the process to reprogram somatic cells to iPSCs. They have also shown that the blocking or inhibition of the interaction of Netrin-1 with its DCC receptor results in a drop in the efficacy of reprogramming somatic cells to iPSCs compared with a reference process without the addition of Netrin-1 or inhibitor of the Netrin-1/DCC interaction.

Therefore the present invention, which sets out to promote the survival of somatic cells during cell reprogramming, can be implemented irrespective of the reprogramming method used.

In the invention Netrin-1 preferably refers to human Netrin-1 and in particular to the polypeptide corresponding to the sequence of 604 amino acids below such as deposited with the Genbank base under access number NP_004813, on 17 Nov. 2006, or to its mature form which corresponds to the amino acids 25 to 604 of this sequence.

| | mmravweala alaavaclvg avrggpglsm fagqaaqpdp csdenghprr cipdfvnaaf |
|---|---|
| 61 | gkdvrvsstc grpparycvv sergeerlrs chlcnasdpk kahppafltd lnnphnltcw |
| 121 | qsenylqfph nvtltlslgk kfevtyvslq fcsprpesma iyksmdygrt wvpfqfystq |
| 181 | crkmynrphr apitkqneqe avctdshtdm rplsggliaf stldgrpsah dfdnspvlqd |
| 241 | wvtatdirva fsrlhtfgde neddselard syfyaysdlq vggrckcngh aarcvrdrdd |
| 301 | slvcdcrhnt agpecdrckp fhydrpwqra tareanecva cncnlharrc rfnmelykls |
| 361 | grksggvcln crhntagrhc hyckegyyrd mgkpithrka ckacdchpvg aagktcnqtt |
| 421 | gqcpckdgvt gitcnrcakg yqqsrspiap cikipvappt taassveepe dcdsyckask |
| 481 | gklkinmkky ckkdyavqih ilkadkagdw wkftvniisv ykqgtsrirr gdqslwirsr |
| 541 | diackcpkik plkkylllgn aedspdqsgi vadksslviq wrdtwarrlr kfqqrekkgk |
| 601 | ckka |

The present invention also covers the use of amino acids sequences of Ntn1, a precursor of 604 amino acids (aa) or mature form (aa25-aa604) with minor modifications such as conservative substitutions for example, provided that these do not significantly affect the action of Netrin-1 in preventing or blocking cell death induced by its receptors and in particular by DCC.

The Netrin-1 contained in the culture medium at least at the start of cell reprogramming process may be natural or recombinant, preferably recombinant. Advantageously the culture medium contains the recombinant Netrin-1 commercially available from Adipogen (ref: AG-40B-0040-0000).

It can be added directly to the culture medium or else produced by cells overexpressing the gene encoding Nerin-1 and which are co-cultured with the somatic cells during reprogramming. Netrin-1 can also be expressed by the somatic cells themselves, in particular by transfection.

Similarly, and as described above for Netrin-1, an analogue of Netrin-1 can be present in the culture medium either directly or produced by cells. By analogue of Netrin-1 is meant any substance capable of mimicking the biological activity of Netrin-1 i.e. any substance capable of applying the biological activity of Netrin-1 and preferably any substance capable of preventing or blocking cell death induced by DCC or UNC5s receptors, in particular cell death induced by the DCC receptor. These analogues are therefore agonists of Netrin-1 that are able to attach to DCC or UNC5s receptors for example or to induce oligomerisation of these receptors in particular of DCC, and to prevent the inducing of cell death mediated by these receptors. These analogues can be selected using any method known to the person skilled in the art and allowing determination in particular of the capability of a substance to attach to DCC or UNC5s receptors, said method possibly comprising an immunoenzymatic assay of ELISA type in particular. These analogues can also be selected using any known method allowing the detection of the capability of a substance to inhibit the attaching of Netrin-1 to the DCC receptor or to the UNC5s receptors, preferably via competitive or non-competitive inhibition of the binding of Netrin-1 to these receptors, more preferably their capability for competitive inhibition of the binding of Netrin-1 to these receptors. These analogues can further be selected using any method allowing determination of the capability of a substance to prevent or block cell death induced by the DCC receptor or by the UNC5s receptors, examples of such methods being described in this present patent application. Among the examples of Netrin-1 analogues, particular mention can be made of agonist antibodies of the DCC or UNC5s receptors, polypeptide fragments of Netrin-1 capable of preventing or blocking cell death mediated by DCC or UNC5s. In the method of the present invention the culture medium of the somatic cells subjected to cell reprogramming may therefore contain one or more fragments of Netrin-1, and in particular fragments of the amino acid sequence deposited with the Genbank base under access number NP_004813, 17 Nov. 2006, provided that this or these fragments preserve the property of inhibiting or blocking cell death mediated by these receptors and in particular by DCC. Amongst these fragments, fragments are particularly cited having at least 30 amino acids, or at least 40 amino acids, or at least 50 amino acids or at least 60 amino acids.

Methods allowing measurement of cell death inhibition mediated by DCC or UNC5s are well known to the person skilled in the art.

Particularly advantageously Netrin-1 or the analogue of Netrin-1 is added directly to the culture medium.

The effect of Netrin-1 addition on the efficacy of the method is dependent on the dose added, the higher the dose the better the result obtained. In the assays conducted by the inventors the best result was obtained with a Netrin-1 concentration of 900 ng/ml. It is within the reach of the person skilled in the art to select the concentration of Netrin-1 or Netrin-1 analogue that is best adapted for the method to be implemented taking into account both improved efficacy but also the cost price of the method regarding the added amount of Netrin-1 or Netrin-1 analogue.

Advantageously the concentration of Netrin-1 contained in the culture medium is at least 100 ng/ml, preferably 150 ng/ml or higher.

When using analogues of Netrin-1, the person skilled in the art will be able to select the appropriate amount of this analogue to obtain the same efficacy as with Netrin-1.

Netrin-1 or the analogue of Netrin-1 is advantageously contained in the culture medium of somatic cells in the process of being reprogrammed for at least the first 7 days effective from initiation of the reprogramming process (D0). The inventors have notably shown that it is not necessary to extend the presence of Netrin-1 beyond this period as no additional advantage is achieved in terms of efficient reprogramming to iPS cells.

In relation to the mode used for addition of Netrin-1 or Netrin-1 analogue to the culture medium, the person skilled in the art will be able to adapt the frequency of this addition taking into account the stability, availability of Netrin-1 or Netrin-1 analogue in the culture medium used.

Advantageously when Netrin-1 is added directly to the culture medium it is added every day, in particular at a concentration of at least 100 ng/ml, especially whenever it is necessary to the change the culture medium during the reprogramming process.

The present invention can be implemented irrespective of the cell reprogramming process used.

The reprogramming process used may comprise the expression in the somatic cells of one or more stem cell pluripotency factors selected from the group consisting of Oct, Sox, Klf and Myc.

Advantageously, the invention is implemented in a reprogramming process comprising even consisting of the expression of Oct4, Sox2, Klf4 and c-Myc transcription factors also known as an OSKM cocktail, which is currently the process most frequently used by the person skilled in the art.

These stem cell pluripotency factors can be expressed in the somatic cells by means of different techniques such as viral infection, transfection in particular using liposomes, electroporation, membrane protein permeability, etc.

Advantageously these stem cell pluripotency factors are added via a viral infection step of the somatic cells, using viruses allowing the expression of nucleic acid sequences encoding these factors and selected in particular from among lentiviruses, retroviruses, adenoviruses, Sendai viruses. These methods are well known to the person skilled in the art and are described in particular in Gonzalez et al., Nat Rev Genet 2011, 12:231-242; Zhou et al., Stem Cells 2009, 27:2667-2674; Weltner et al., J Virol 2012, 86:4463-4467; Fusaki et al., Proc Jpn Acad Ser B Phys Biol Sci 2009, 85:348-362; Nishimura et al., J Biol Chem 2011, 286:4760-4771.

The method of the invention may further comprise a step to add a reporter system to the somatic cells to indicate the efficacy of iPS cells generation and production, selected in particular from among fluorescent systems such as GFP, colorimetric systems, antibiotic resistance systems, etc.

According to one particularly preferred embodiment the present invention is implemented using a cell reprogramming process that is conducted without genomic integration and/or without expression of oncogenes.

Different types of somatic cells can be reprogrammed to iPSCs in the method of the present invention and in particular fibroblasts, keratinocytes, T cells, hepatocytes, umbilical cord cells, adipose cells, intestinal cells and blood cells, advantageously fibroblasts.

The present invention finds particular application in the preparation of mammalian iPSCs. Therefore the somatic cells used in the method of the invention can be selected from the group consisting of rat, mouse, rabbit, pig, sheep, goat, cow, monkey and human. Advantageously the somatic cells used are human somatic cells and in particular human fibroblasts. According to one particular aspect, the somatic cells used are human intestinal epithelial cells.

According to a second aspect the present invention also relates to the use of Netrin-1 or analogue of Netrin-1 to prepare induced pluripotent stem cells (iPSCs) via culture of somatic cells subjected to a cell reprogramming process, in particular to improve the efficacy of cell reprogramming of somatic cells to iPSCs.

All the particular and advantageous embodiments mentioned above relating to the method for producing iPSCs are also advantageous implementations with regard to the use of Netrin-1 or analogue of Netrin-1 for iPSC production.

### DESCRIPTION OF THE FIGURES

FIG. 1 illustrates the expression level of Netrin-1 and its DCC receptor (FIG. 1A) and its UNC5B and UNC5C receptors (FIG. 1B) during MEF reprogramming (Mouse Embyronic Fibroblasts), at days 0, 2, 4 and 6 after infection with OSKM-encoding retroviral particles.

FIG. 2 illustrates the effect of inactivation of the expression of Netrin-1 on reprogramming of MEFs infected with OSKM lentiviral particles (FIG. 2A) and on the reprogramming of intestinal epithelium caused to reprogram via treatment with doxycycline (FIG. 2B).

FIG. 3 illustrates the effect of increasing doses of Ntn1 on the efficacy of cell reprogramming (FIG. 3A) and on the reprogramming of intestinal epithelium caused to reprogram via treatment with doxycycline (FIG. 3B).

FIG. 4 illustrates the effect of the sequential addition of Ntn1 on the efficacy of cell reprogramming (D0-7: 7 first days, D7-14: from Day 7 to Day 14 and D0-14: 14 first days).

FIG. 5 illustrates the effect of Ntn1-blocking antibodies on the efficacy of cell reprogramming (D0-7: 7 first days, D7-14: Day 7 to Day 14 and D0-14: 14 first days).

FIG. 6 illustrates the effect of inactivation of the expression of the DCC receptor (A) and of Ntn1 expression (B) on the efficacy of cell reprogramming.

FIG. 7 illustrates phosphatase alkaline activity (A) and DESeq hierarchical clustering analysis (B) of clones of iPS cells derived under “control” conditions or in the presence of recombinant Netrin-1.

FIG. 8 illustrates analysis of major chromosomal disorders between the control iPSCs lines (white histogram) and derivatives in the presence of Ntn1 (black histogram).

FIG. 9 illustrates the histological analysis of teratomas induced by in vivo injection of iPSCs clones derived in the presence of Ntn1 (Scale size: 100 μm).

FIG. 10 illustrates the effect of recombinant Netrin-1 (black histogram) on the emergence of positive SSEA4 colonies from fibroblasts of human foreskin replaced in culture on fibroblasts treated with mitomycin C, 7 days after infection (bar scale corresponds to 200 μm) relative to the control (white histogram).

### EXAMPLES

### Cell Culture and RNAi Assays

Mouse embryonic fibroblasts (MEF) were derived from embryos at E13.5 from different strains. The pre-iPS, iPS and ES cells were cultured on irradiated or on-gelatin MEFs as described previously. The 293T and plat-E cells were cultured in DMEM supplemented with 10% FCS and penicillin/streptomicin. The iPS cells were cultured in KSR+LIF or KSR 2i+LIF medium. shRNA assays were conducted using pLKO.1 vectors and siRNA by Sigma (SHCLNG-NM_008744 for Ntn1, SHCLNG-NM_007831 for Dcc and EMU022741 for Mbd3).

Antibodies:

The antibodies used in this assay for immunofluorescence and FACS were the following: anti-Oct4 (Santa Cruz, C10), anti-Netrin-1 (R&D Systems, mab1109), anti-thy1 (Ebiosciences, 53-2.1), anti-SSEA1 (Stem cell technologies, 60060PE) and NL493 conjugated anti-SSEA4 antibody (R and D Systems SC023).

Recombinant Netrin-1:

The recombinant Netrin-1 used is commercially available from Adipogen (ref: AG-40B-0040-0000).

Retroviral and Lentiviral Production.

The plat-E cells were used to produce retroviral particles from pMX-s vectors encoding the cDNA of Oct4, Sox2, Klf4 and c-Myc, as previously described by the inventors. Viral particles encoding mcherry were used to monitor the efficacy of MEF infection. 293T cells were used to produce the lentiviral particles.

Generation of Mouse iPS Cells.

MEF cells were passaged in 6-well dishes. 12 hours later these cells were infected with equivalent amounts of each retroviral particle in the presence of Polybrene at a concentration of 8 μg/ml Polybrene. The culture medium was replaced 24 hours after infection and 48 hours after infection, the MEFs were collected and replaced in culture on irradiated fibroblasts in culture medium for iPS cells. The culture medium was then replaced every day with fresh medium and the emergent colonies were counted and collected 12-14 days post-infection. The intestinal epithelium was separated and the epithelium fragments placed in culture on an irradiated feeder cells layer. The pluripotent reprogramming process here was induced by treating the cells with doxycycline (2 μg/ml, for 14 days) since mice having an inducible expression cassette of Oct4, Sox2, Klf4 and c-Myc reprogramming factors were used.

Generation of Human iPS Cells.

The assays were performed with 5×10⁵ human foreskin cells (HFF, Millipore) placed in culture in a well of a 6-well plate and in the presence of FibroGRO™-LS culture medium (Millipore) until being replaced in culture on fibroblasts. The cells were infected overnight with 4 Sendai viruses respectively encoding Oct4, Sox2, Klf4 and c-Myc (Cytotune™, Life Technologies) at a MOI of 3. The culture medium was changed the following day and replaced by medium containing or not containing recombinant Netrin-1 at 150 or 750 ng/mL. The medium was then changed every day. 7 days after infection the cells were separated using TrypLE Express (Life Technologies), counted and replaced in culture on MEFs treated with mitomycin C (Sigma Aldrich) at a cell density of 5×10, 1×10⁵ or 2×10⁵ HFF per plate. The following day the medium was replaced by DMEM/F12 (Life Technologies) supplemented with 20% PluriQ™ Serum Replacement (GlobalStem), 0.1 mmol/L non-essential amino acids, 1 mmol/L L-glutamine, 0.1 mmol/L 2-mercaptoethanol, penicillin/streptomycin (Life Technologies), 12.5 ng/ml human basic fibroblast growth factor (Milytenyi Biotec) in the presence or not in the presence of recombinant Netrin-1. The medium was then changed every day. At Day 26 reprogrammed colonies were manually sub-cultured and transferred onto new MEFs treated with mitomycin C for amplification.

RNA-Sequencing:

RNA quality was analysed using a Bioanalyser (Agilent). The libraries were constructed and sequenced on HiSeq by Illumina 2000.

Formation of Teratomas:

5×10⁶ iPS cells were injected beneath the renal capsules of immunodeficient mice (SCID) aged 7 weeks (CB17/SCID, Charles River). After 3 weeks, the mice were euthanized and the tumours surgically removed and fixed in 4% formalin or in PFA for cryosections. The same procedure was used with the injection of 5×10⁵ iPS cells in the testicles.

### Example 1

### Study on the Expression of Netrin-1 During the Reprogramming of Murine Somatic Cells

Murine embryonic fibroblasts (MEF) were infected with OSKM-encoding retroviral particles for 6 days. The expression of Netrin-1, of the receptors DCC, UNC5B and UNC5C were measured over time (D0, D2, D4 and D6 for Netrin-1 and DCC, and D0, D3 and D6 for UNC5B and UNC5C).

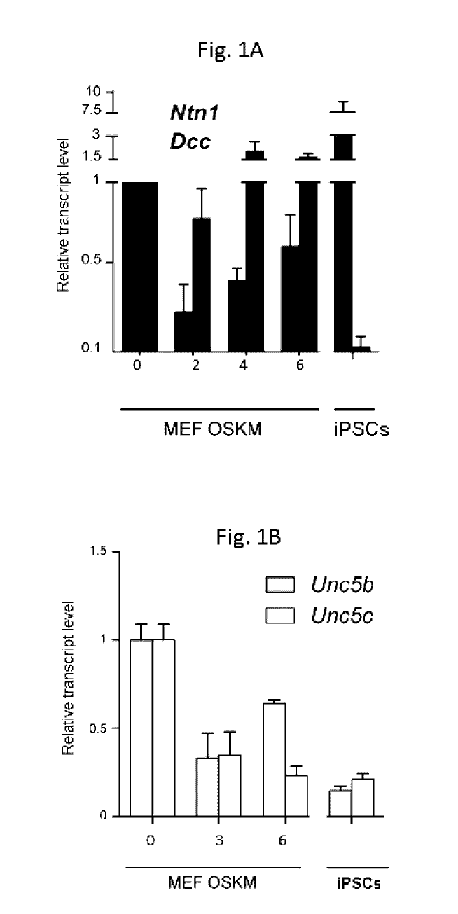

The results are given in appended FIG. 1. q-RTPCR analyses show the expression profiles of Ntn1, DCC, UNC5B and UNC5C over the first 6 days of cell reprogramming to the pluripotent state and in established iPS cells. The data are normalised against the RS17 and L19 housekeeping genes, and represented relative to the expression level in MEFs and correspond to the mean±SEM of 3 independent assays.

They show a two-phase expression of Netrin-1 with a strong reduction over the first 6 days of the cell reprogramming process followed by reactivation in the iPS cells. In parallel, the expression of the DCC receptor remains stable (FIG. 1A), whereas the expression of the other receptors such as UNC5B and UNC5C is rapidly repressed (FIG. 1B). Therefore the expression of Ntn1 is reduced whilst that of its DCC receptor is maintained during the initial phases of cell reprogramming. The same expression profile was observed with a Dox-inducible system during MEF reprogramming (results not appended).

### Example 2

### Effect of Inactivation of Netrin-1 on the Efficacy of Reprogramming Murine Somatic Cells

The effect of inactivation of the expression of Ntn1 on cell reprogramming was analysed by quantification of positive alkaline phosphatase colonies or positive Nanog. Positive AP cells are characterized by a test evidencing enzymatic activity of the cells; positive Nanog cells are characterized by immunofluorescence using Nanog-specific antibodies. MEFs were infected with lentiviral particles coding for three different shRNAs which target Ntn1, two days before OSKM transduction. The number of colonies produced in the control MEFs infected with shscrambled particles comprising a control shRNA not targeting Netrin-1 was brought to 100% for each assay. Three different shRNAs were used (Ntn1 sh#1, Ntn1 sh#2 and Ntn1 sh#3). The data given correspond to the mean±SEM of three independent assays performed with different batches of MEF and viral particles.

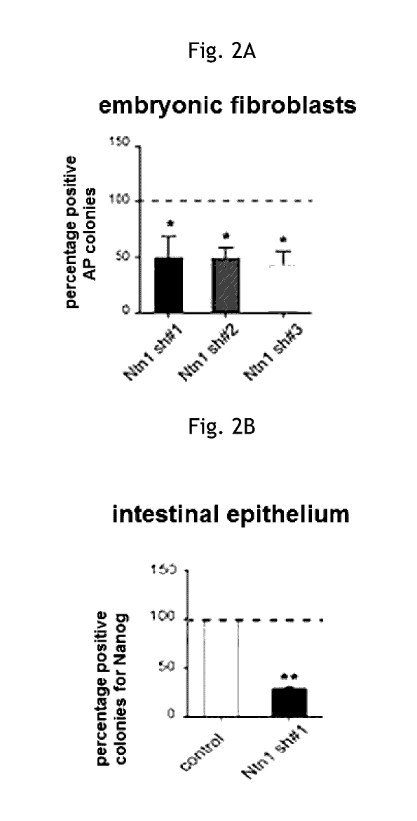

The results are grouped together in appended FIG. 2. They show that Netrin-1 depletion leads to a significant decrease in cell reprogramming as indicated by the number of AP-positive colonies from embryonic murine fibroblasts (FIG. 2A).

Similar results were obtained using another type of somatic cell: intestinal epithelium (FIG. 2B).

### Example 3

### Effect of Compensation for Ntn-1 Deficiency on Cell Reprogramming

Different concentrations of recombinant Netrin-1 were added daily to the cells (0.15 μg/ml; 1 μg/ml and 4 μg/ml) and the number of positive AP colonies were counted 12-14 days post-OSKM infection.

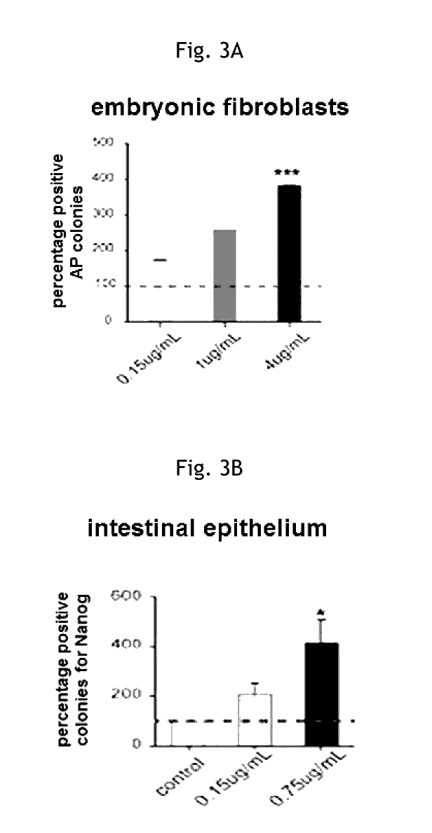

The results are grouped together in appended FIG. 3. They show that the exogenous providing of recombinant Netrin-1 improves the efficacy of cell reprogramming in dose-dependent manner; the highest dose allowing an increase in the number of AP-positive colonies by a factor of 4 from embryonic murine fibroblasts (FIG. 3A).

Similar results were obtained using another type of somatic cell, intestinal epithelium (FIG. 3B).

To examine whether or not Ntn1 is required throughout the entire duration of the cell reprogramming process, the effect of sequential treatments of recombinant Netrin-1 on cell reprogramming was examined by quantification of AP-positive colonies. The number of colonies obtained without treatment was brought to 100% for each individual assay. The data given correspond to the mean±SEM of three independent experiments. Student's T-test was used for statistical analyses.

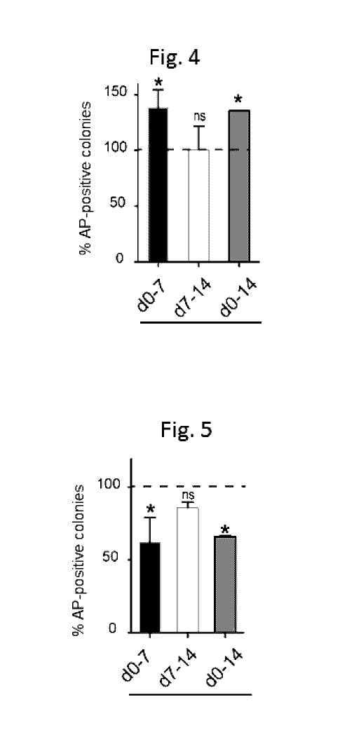

The results are grouped together in appended FIG. 4. They show that the addition of recombinant Ntn1 over the first 7 days of the cell reprogramming process (D0-7) is sufficient to obtain the effect of improved reprogramming. On the contrary, the treatment of cells from Day 7 to Day 14 of reprogramming (D7-14) has no positive impact in terms of efficacy.

These results show the positive impact of the early presence of Netrin-1 when reprogramming somatic cells to iPS cells which was confirmed by the results obtained with the same experiments performed with Ntn-1 blocking antibodies (FIG. 5).

### Example 4

### Study on the Impact of Reduced DCC Expression, with Greater or Lesser Ntn1, During the Early Phase of Cell Reprogramming

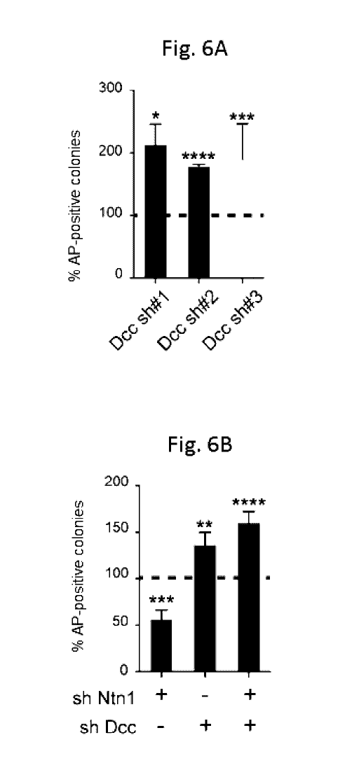

The effect of inactivation of the expression of the DCC receptor was examined using a shRNA lentiviral strategy via quantification of positive alkaline phosphatase colonies (AP-positives); the results are grouped together in appended FIG. 6 in which the number of colonies produced in MEFs infected with shscrambled particles under control conditions was brought to 100% for each experiment. Three different shRNAs were used (DCC sh#1, DCC sh#2 and DCC sh#3). The data given correspond to the mean±SEM of three independent experiments performed with different batches of MEF and viral particles.

The results show that inactivation of DCC improves cell reprogramming (FIG. 6A). In addition, the effect of Ntn1 inactivation on cell reprogramming is reversed by inactivation of DCC (FIG. 6B), showing the Ntn1/DCC pair to be a novel key mediator for cell reprogramming of somatic cells to iPS cells.

### Example 5

### Use of Ntn1 as Soluble Factor to Improve the Reprogramming of Murine and Human Somatic Cells to iPSCs

Murine Oct4-GFP iPSCs clones derived under standard conditions (control) or in the presence of 600 ng/ml Ntn1 during the 7 first days of cell reprogramming were individually passaged and placed in culture.

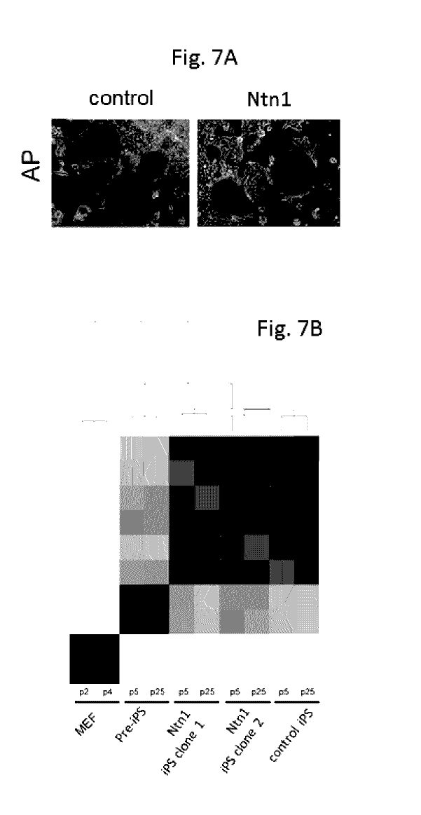

Measured AP morphology and activity were similar between the control iPSCs clones and in the presence of Ntn1 (FIG. 7A), which was confirmed by the close correlation revealed by DESeq hierarchical clustering analysis of MEF Oct4-GFP, pre-iPS and iPS cells derived under “control” conditions or in the presence of recombinant Netrin-1 at two different passages: p5 and p25 (FIG. 7B).

The results obtained with reprogramming protocols using different stem cell pluripotency factors such as Pou5f1, Sox2, Klf4, Nanog, Fgf4, Esrrb, Utf1 and Dppa3 allowed the same conclusions to be drawn (results not appended).

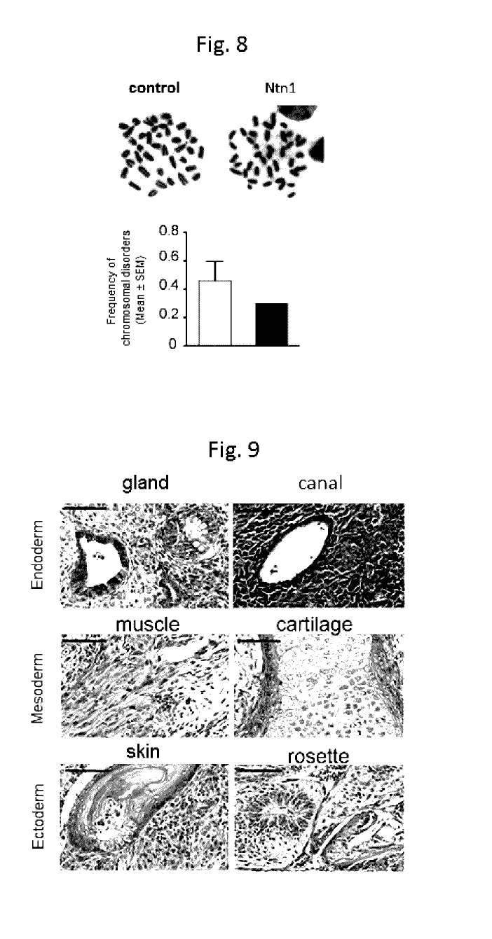

After 40 passages in culture, the quantification of major chromosomal disorders did not reveal any significant difference between the control iPSCs lines and those derived in the presence of recombinant Ntn1, as shown in the results grouped together in FIG. 8.

Therefore the reprogramming of murine somatic cells to iPS cells in the presence of Ntn1 has no harmful effect on chromosomal stability.

The differentiation potential of murine iPSCs derived in the presence of Ntn1 was examined in vivo by histological analysis of teratomas obtained from the injection of iPSCs produced in the presence of recombinant Netrin-1 (FIG. 9) and in vitro via the formation of embryoid bodies (EB), demonstrating the emergence of derivatives of the three types of embryonic layers (endoderm, mesoderm and ectoderm) (results not appended). It was also confirmed that the murine iPS cells derived in the presence of Ntn1 are suitably integrated in the host blastocysts (results not appended).

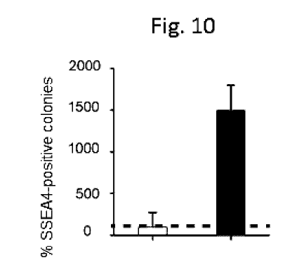

Finally the effect of recombinant Ntn1 on the reprogramming of human foreskin fibroblasts was analysed after marking living cells with an antibody directed against SSEA4. The results grouped together in appended FIG. 10 show that treatment with recombinant Ntn1 (0.15 μg/ml) induces an increase in the efficacy of cell reprogramming by a factor of 15.

To conclude, the reprogramming of somatic cells to iPS cells performed in the presence of Netrin-1 during the early phases of the cell reprogramming process improves the efficacy of iPS cells generation, in particular murine and human.

1. A method for producing induced pluripotent stem cells (iPSCs) via culture of somatic cells subjected to a cell reprogramming process, comprising the step of culturing, the somatic cells in the presence of Netrin-1 or an analogue of Netrin-1 at least at the start of the cell reprogramming process, said analogue of Netrin-1 being capable of mimicking the biological activity of Netrin-1.

2. The method for producing induced pluripotent stem cells (iPSCs) according to claim 1, wherein said analogue of Netrin-1 is selected from the group consisting of agonist antibodies of the DCC or UNC5s receptors and polypeptide fragments of Netrin-1 capable of preventing or blocking cell death mediated by DCC or UNC5s.

3. The method according to claim 1, wherein the somatic cells subjected to a cell reprogramming process are cultured in a culture medium comprising at least 100 ng/ml Netrin-1.

4. The method according to claim 1, wherein the somatic cells subjected to a cell reprogramming process are cultured for at least the first 7 days effective from initiation of the reprogramming process in a culture medium comprising Netrin-1 or analogue of Netrin-1.

5. The method according to claim 1, wherein Netrin-1 or analogue of Netrin-1 is added to the culture medium every day at a concentration of at least 100 ng/ml.

6. The method according to claim 1, wherein the reprogramming process comprises the expression of one or more stem cell pluripotency factors in the somatic cells selected from the group consisting of Oct, Sox, Klf and c-Myc.

7. The method according to claim 1, wherein the reprogramming process comprises the expression of Oct4, Sox2, Klf4 and c-Myc transcription factors.

8. The method according to claim 1, wherein it further comprises a step to add a reporter system to the somatic cells to indicate the efficacy of iPS cells generation and production.

9. The method according to claim 1, wherein the cell reprogramming process is conducted without genomic integration and/or without expression of oncogenes.

10. The method according to claim 1, wherein the somatic cells are selected from the group consisting of fibroblasts, keratinocytes, T cells, hepatocytes, umbilical cord cells, adipose cells, intestinal cells and blood cells.

11. The method according to claim 1, wherein the somatic cells are somatic cells of mammals selected in particular from the group consisting of rat, mouse, rabbit, pig, sheep, goat, cow, monkey and human, advantageously they are human somatic cells.

12. A method for improving the efficacy of cell reprogramming of somatic cells to iPS cells comprising the step of using Netrin-1 or analogue of Netrin-1 to prepare induced pluripotent stem cells (iPSCs) via culture of somatic cells subjected to a cell reprogramming process.

13. The method according to claim 12, wherein said analogue of Netrin-1 is selected from the group consisting of agonist antibodies of the DCC or UNC5s receptors and polypeptide fragments of Netrin-1 capable of preventing or blocking cell death mediated by DCC or UNC5s.

14. The method according to claim 4 wherein the reprogramming process comprises the expression of one or more stem cell pluripotency factors in the somatic cells selected from the group consisting of Oct, Sox, Klf and c-Myc.
